(19) United States
(12) Patent Application Publication
(10) Pub. No.: US 2024/0224972 A1
(43) Pub. Date: Jul. 11, 2024

(54) DEVICE FOR PREVENTING BIOFOULING OF SHIP AND MANUFACTURING METHOD THEREOF

(71) Applicant: Proxihealthcare Inc., Ulsan (KR)

(72) Inventor: Young Wook Kim, Seoul (KR)

(21) Appl. No.: 18/528,679

(22) Filed: Dec. 4, 2023

Related U.S. Application Data
(63) Continuation of application No. PCT/KR2021/019107, filed on Dec. 15, 2021.

(30) Foreign Application Priority Data
Jun. 4, 2021 (KR) 10-2021-0073009

(51) Int. Cl. A01M 1/22; A61L 2/03

(52) U.S. Cl. CPC A01M 1/22; A61L 2/03

(57) ABSTRACT

The present invention relates to a biofouling preventing device for ships and a method for manufacturing same. The biofauling preventing device for ships includes: an electrode disposed on a surface area of a hull and configured to supply with a driving signal to provide an electromagnetic wave corresponding to the driving signal; and a signal supply configured to supply the driving signal to the electrode.

### CROSS REFERENCE TO RELATED APPLICATIONS

This application is a Bypass Continuation of International Application No. PCT/KR2021/019107, filed on Dec. 15, 2021, and claims priority from and the benefit of Korean Patent Application No. 10-2021-0073009, filed on Jun. 4, 2021, each of which is incorporated by reference for all purposes as if fully set forth herein.

### BACKGROUND

### Field

The present invention relates to a biofouling prevention device for ships using a special microcurrent electromagnetic wave, and a method of manufacturing the same.

### Discussion of the Background

Biofouling is caused by marine organisms such as seaweed, sphagnum moss, and shellfish that live in the ocean and adhere to the surface of ships, increasing the surface drag of the ships, reducing the speed at which the ship can operate, increasing fuel consumption, and increasing overall operating costs.

In addition, marine organisms attached to hulls causes the paint on the hulls to flake off, which is a factor in hull corrosion, and recently, the migration of ship-attached organisms has been identified as a cause of ecosystem disturbance.

The removal of marine organisms attached to the hulls has typically been done by divers using brushes or underwater robots. However, these conventional methods are inefficient, requiring large numbers of personnel, long working hours and excessive costs.

### SUMMARY

An object of the present invention, which is made to solve the above-mentioned problem, is to provide a biofouling preventing device for ships which is capable of effectively preventing biofouling of ships by using an electromagnetic wave, and a method of manufacturing the same.

In addition, another object of the present invention is to provide a biofouling preventing device for ships which enhances the removal effect of biofilms causing biofouling by using a driving signal generated by mixing an AC signal and a DC signal, and a method of manufacturing the same.

In addition, another object of the present invention is to provide a biofouling preventing device for ships which is capable of customized management by area by setting different characteristics of the electrodes for different areas of the ships, and a method of the same.

In addition, another object of the present invention is to provide a method of manufacturing a biofouling preventing device that facilitates the formation of large-area electrodes by an electrode patterning process using a shadow mask.

A biofauling preventing device for ships according to an embodiment of the present invention may include: an electrode disposed on a surface area of a hull and configured to supply with a driving signal to provide electromagnetic waves corresponding to the driving signal; and a signal supply configured to supply the driving signal to the electrode.

In addition, the driving signal may be generated by mixing an AC signal and a DC signal.

In addition, the signal supply may be configured to modify at least one of the characteristics of the driving signal in response to an external input.

In addition, the characteristics of the driving signal may include an amplitude and a DC offset.

In addition, the biofauling preventing device may further include a protective layer disposed on the electrode.

In addition, the surface area may include a first surface area and a second surface area, and the electrode may include a first electrode disposed on the first surface region and a second electrode disposed on the second surface region.

In addition, the first electrode may have a width different from the second electrode.

In addition, the signal supply may supply a first driving signal and a second driving signal, which are a mixture of the AC signal and the DC signal, to the first electrode and the second electrode, respectively, and at least one of an amplitude and a DC offset of the first driving signal is set to be different from at least one of a corresponding amplitude and a DC offset of the second driving signal.

In addition, the biofauling preventing device may further include a resistance measuring device configured to measure the resistance of the electrode, wherein the signal supply may modify at least one of an amplitude and a DC offset of the drive signal in response to a resistance value measured by the resistance measuring device.

A method of manufacturing a biofouling preventing device for a ship according to an embodiment of the present invention may include: placing a mask on a surface area of a hull; supplying a conductive material to the side of the mask to form an electrode patterned in a predetermined shape on the surface area; and forming a protective layer on the electrode.

In addition, the method may further include installing a signal supply configured to supply a driving signal to the electrode, wherein the driving signal may be generated by mixing an AC signal and a DC signal.

A method of manufacturing a biofouling preventing device for a ship according to an embodiment of the present invention may include: placing a first mask on a first surface area of a hull; supplying a conductive material to the side of the first mask to form a first electrode patterned in a predetermined shape on the first surface area; placing a second mask on a second surface area of the hull; and supplying a conductive material to the side of the second mask to form a second electrode patterned in a predetermined shape on the second surface area, wherein the first electrode and the second electrode have at least one of a physical characteristic and an electrical characteristic that are set to be different.

In addition, the physical characteristic may include at least one of a width, a thickness, and a density of the first electrode and the second electrode, and the electrical characteristic may include at least one of an amplitude and a DC offset of the driving signal supplied to the first electrode and the second electrode.

According to an embodiment of the present invention, it is possible to provide a biofouling preventing device for ships which is capable of effectively preventing biofouling of ships by electromagnetic waves, and a method of manufacturing the same.

In addition, according to an embodiment of the present invention, it is possible to provide a biofouling preventing device for ships which enhances the removal effect of biofilm causing biofouling by using the driving signal generated by mixing an AC signal and a DC signal, and a method of manufacturing the same.

In addition, according to an embodiment of the present invention, it is possible to provide a biofouling preventing device for ships, which is capable of customized management on an area-specific basis by setting different characteristics of the electrodes for different areas of the ships, and a method of manufacturing the same.

In addition, according to an embodiment of the present invention, it is possible to provide a method of manufacturing a biofouling preventing device that facilitates the formation of large-area electrodes by an electrode patterning process using a shadow mask.

### BRIEF DESCRIPTION OF THE DRAWINGS

FIG. 1 is a diagram illustrating a ship according to an embodiment of the present invention.

FIG. 2 is a diagram illustrating a biofouling preventing device for ships according to an embodiment of the present invention.

FIG. 3 is a cross sectional view illustrating electrodes and a protective layer according to an embodiment of the present invention.

FIGS. 4A and 4B are diagrams explaining a biofilm removal effect by a driving signal generated by mixing an AC signal and a DC signal.

FIG. 5 is a diagram illustrating a signal supply module according to an embodiment of the present invention.

FIGS. 6A to 6C are diagrams illustrating the waveforms of signals according to an embodiment of the present invention.

FIG. 7A is a diagram illustrating a controller and a signal supply module according to an embodiment of the present invention, and FIG. 7B is a diagram illustrating a resistance measuring device, a controller, and a signal supply module according to an embodiment of the present invention.

FIG. 8A is a diagram illustrating a first surface region and a second surface region of an embodiment of the present invention, FIG. 8B is a diagram illustrating a partial cross-section of the first surface region and the second surface region illustrated in FIG. 8A, and FIG. 8C is a diagram illustrating a first surface region and a second surface region of another embodiment of the present invention.

FIGS. 9A to 9C are diagrams illustrating a method of manufacturing a biofouling preventing device for ships according to an embodiment of the present invention.

FIGS. 10A to 10C are diagrams illustrating a method of manufacturing a biofouling preventing device according to another embodiment of the present invention.

### DETAILED DESCRIPTION

In the following, embodiments related to the present invention are illustrated in the drawings and described in detail by way of a detailed description. However, the present invention is not limited to the embodiments disclosed below and may be implemented in various different forms. Also, it should be understood that all modifications, equivalents, or replacements thereof are included within the subject matter and scope of the present invention.

In describing elements of the present invention, terms such as first, second, A, B, (a), and (b) may be used. These terms are only used to distinguish one element from other elements, and the nature, sequence, or order of that element is not limited by the term. Further, it should be understood in this specification that if an element is described as being “connected”, “combined”, or “coupled” to/with any other element, the element may be directly connected, combined, or coupled to/with the other element, but another element may also be connected, combined, or coupled between both elements. In the case of being “connected”, “combined”, or “coupled”, it may be understood as being physically or electrically connected, combined, or coupled, but is also electrically “connected”, “combined”, or “coupled”” as needed.

Terms such as “˜ unit”, “˜er”, “part”, and “˜ module” used in this specification refer to a unit that processes at least one particular function or operation, and may be implemented with hardware, software, or a combination thereof. In addition, terms such as “comprise”, “include”, and “have” used in this specification denote the presence of a stated element unless the relevant context clearly indicates otherwise, and do not exclude the presence of or a possibility of addition of one or more other elements.

In addition, it should be clarified that the division of the elements herein is merely based on the primary function performed by each element. That is, two or more elements to be described below may be combined into one element, or one element may be divided into two or more elements according to subdivided functions. It should also be noted that each element described below may, in addition to its primary function, perform some or all of the functions performed by other elements, and that a portion of the primary function of each element may be performed by other elements.

Hereinafter, a biofouling preventing device for ships according to an embodiment of the present invention will be described with reference to drawings related to embodiments of the present invention.

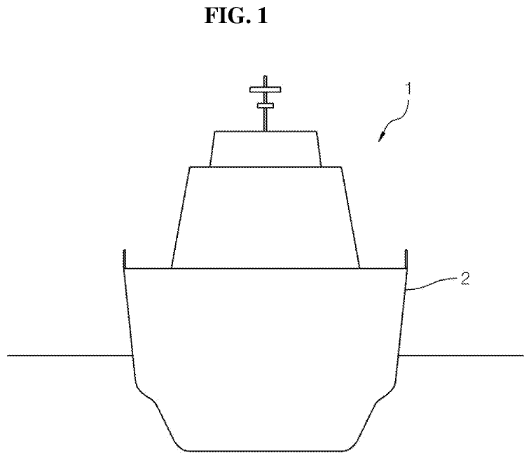

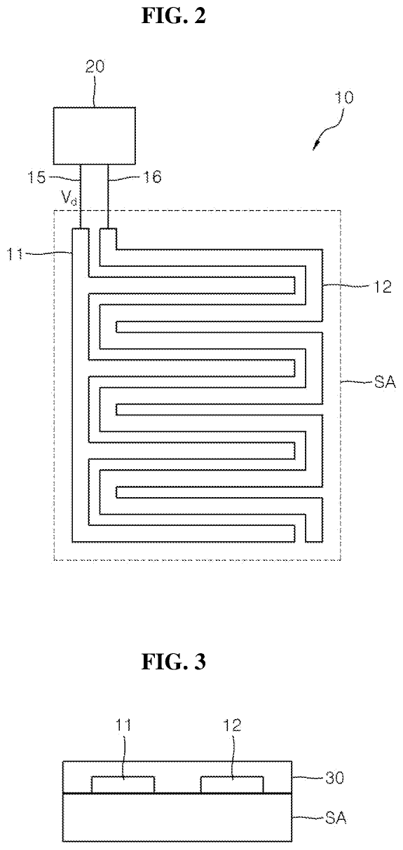

FIG. 1 is a diagram illustrating a ship according to an embodiment of the present invention, FIG. 2 is a diagram illustrating a biofouling preventing device for ships according to an embodiment of the present invention, and FIG. 3 is a cross sectional view illustrating electrodes and a protective layer according to an embodiment of the present invention.

Referring to FIG. 1, a ship 1 have a hull 2 in which a cargo space for loading cargo or the like is formed, and various facilities for operating the ship are provided on the hull 2. Most of these ships 1 are normally operated with the lower part of the hull 2 submerged in the water or remain anchored.

As a result, various fouling organisms may attach to and colonize the hull 2, resulting in biofouling of the hull 2.

It is known that the development of misogynistic organisms is sequential. When the surface of the hull is submerged in seawater, organic substances (which are mostly protein components) are adsorbed to the surface within a few minutes, and bacteria and microalgae attach within a day, forming what is known as a primary biofilm. After about week, macroalgal spores attach and protozoa attach to it and grow, forming what is known as a secondary biofilm. When it is more than two weeks to a month or more after exposure, larvae such as the barnacles, lichen worms, anemones, mollusks, and hornworms, which are commonly visible, attach and form what is known as a tertiary biofilm.

Accordingly, a biofouling preventing device for ships according to an embodiment of the present invention is proposed to minimize the occurrence of biofouling by providing electromagnetic waves specialized for removing the biofilms to the hull 2.

Referring to FIGS. 2 and 3, a biofouling preventing device 10 for ships according to an embodiment of the present invention may include an electrode 11 and a signal supply module 20.

The electrode 11 may be disposed on a surface area SA of the hull 2, and may be supplied with a driving signal Vd to provide electromagnetic waves corresponding to the driving signal Vd.

That is, the electrode 11 may emit the electromagnetic waves to the outside based on the electrical energy of the driving signal Vd, and the electromagnetic waves may inhibit the formation of biofilms on the surface area SA.

In addition, the surface area SA may have a separate auxiliary electrode 12 spaced apart from the electrode 11 to facilitate the generation of the electromagnetic field. For example, the electrode 11 may be set as a positive electrode, and the auxiliary electrode 12 may be set as a negative electrode or ground electrode.

The electrode 11 and the auxiliary electrode 12 may be formed of a material such as, but not limited to, copper, brass, aluminum, conductive polymer, conductive silicon, stainless steel and so on, and may be formed based on metal salt compositions, nanometallic compositions, and conductive compositions suitable for the shadow mask method.

Further, the electrode 11 and the auxiliary electrode 12 may be implemented in the form of a line having a predetermined width, and FIG. 2 illustrates, but is not limited to, an exemplary form in which the electrode 11 and the auxiliary electrode 12 are staggered and engaged with each other.

On top of the surface area SA where the electrode 11 and the auxiliary electrode 12 are located, a protective layer 30 may be formed for protection of the electrode 11 and the auxiliary electrode 12. Such protective layer 30 may be made of a material that is seawater resistant and durable.

The signal supply module 20 is arranged on the ship 1 and may be electrically connected to the electrode 11 and the auxiliary electrode 12 via separate wiring 15 and 16. For example, the signal supply module 20 may supply the driving signal Vd to the electrode 11 via the first wiring 15, and the auxiliary electrode 12 may be supplied with a negative voltage or ground voltage via the second wiring 16.

In particular, the signal supply module 20 may generate the driving signal Vd by mixing an alternating current (AC) signal and a direct current (DC) signal.

Accordingly, the driving signal Vd may include both AC component and DC component, and synergistic effects and resonance may occur by simultaneously applying the AC component and DC component, thereby increasing the removal effect of the biofilms that cause the biofauling.

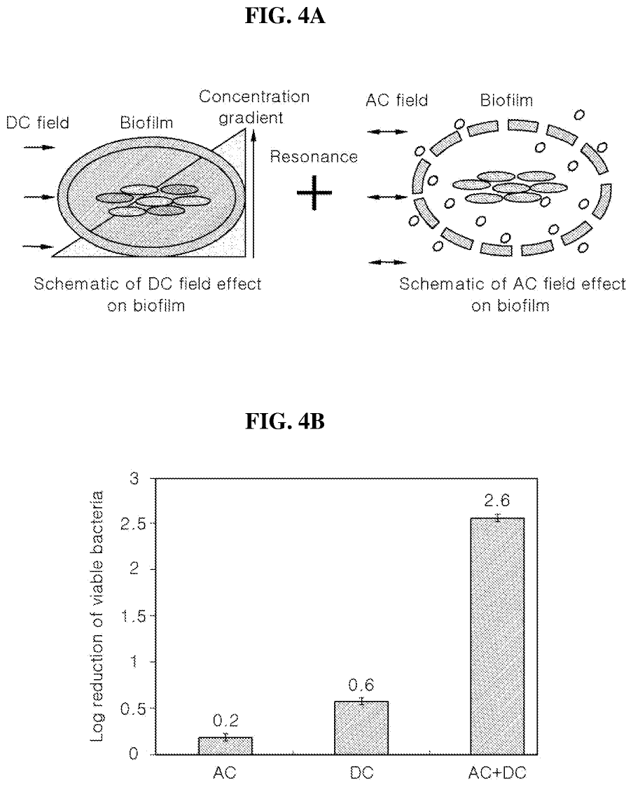

FIGS. 4a and 4b are diagrams explaining the removal effect of a biofilm by the driving signal generated by mixing an AC signal and a DC signal.

Referring to FIG. 4a, an electric field induced by the DC component of the driving signal Vd may increase the structural stress of the biofilms by inducing an imbalance in the local charge distribution, and an electric field induced by the AC component of the driving signal Vd may increase the permeability to the external protector by generating specific vibrations.

The synergistic effect resulting from these AC component and DC component may be observed from FIG. 4b. Specifically, compared to the biofilm removal effect when the electric field induced by the AC component and the electric field induced by the DC voltage are provided separately, it can be seen that the biofilm removal effect is significantly superior when the electric field induced by the AC component and the electric field induced by the DC voltage are provided simultaneously in an overlapping manner.

Since the electric field induced by the DC component and the electric field induced by the AC component in response to the driving signal Vd supplied from the signal supply module 20 according to an embodiment of the present invention may be provided simultaneously by the electrode 11, the above-described enhanced biofilm removal effect may be achieved.

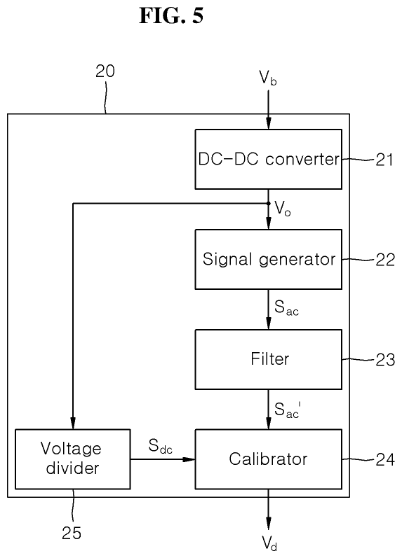

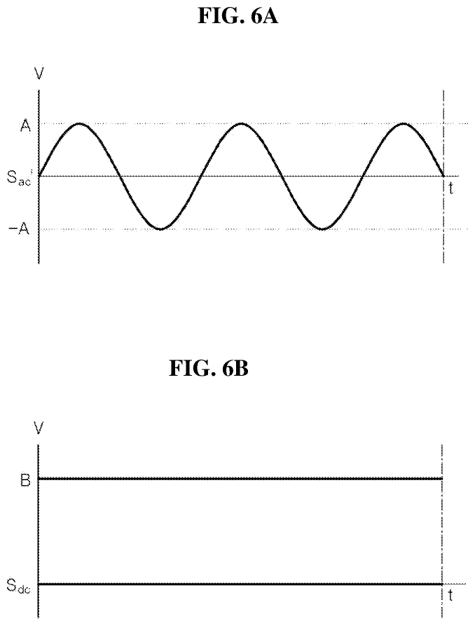

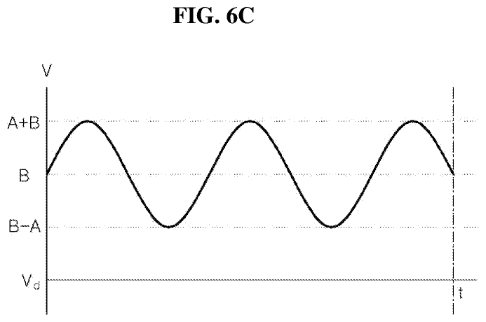

FIG. 5 is a diagram illustrating a signal supply module according to an embodiment of the present invention; and FIGS. 6a to 6c are diagrams illustrating the waveforms of signals according to an embodiment of the present invention. In particular, FIG. 6a shows a filtered AC signal Sac′, FIG. 6b shows a DC signal Sdc, and FIG. 6c shows a driving signal Vd generated by mixing the filtered AC signal Sac′ and the DC signal Sdc.

Referring to FIG. 5, the signal supply module 20 according to an embodiment of the present invention may include a DC-DC converter 21, a signal generator 22, a filter 23, and a calibrator 24, and may additionally include a voltage divider 25.

The DC-DC converter 21 may receive an external voltage Vb, convert the external voltage Vb into an output voltage Vo of a predetermined level, and output it.

The signal generator 22 may operate based on a voltage supplied from the DC-DC converter 21, and may generate an AC signal Sac with a predetermined frequency using the output voltage Vo from the DC-DC converter 21.

The signal generator 22 may be implemented using a known configuration, such as an oscillator, a function generator, or the like, capable of generating an AC signal.

For example, a frequency in the range of 1 KHz to 1000 MHz may be set for the AC signal Sac. This is because when the AC signal Sac is set to a low frequency below 1 KHz, the effect of removing the biofilms will be reduced, and even when the AC signal Sac is set to an ultra-high frequency above 1000 MHz, the effect of removing the biofilms will also be reduced. Meanwhile, the frequency of the AC signal Sac may be set to a frequency in the range of 5 MHz to 15 MHz suitable for biofilm removal.

In addition, the amplitude of the AC signal Sac may be set in the range of, but not limited to, 0.1 mv to 10 V, which is suitable for the prevention of biofouling of the ship 1.

The filter 23 may perform a filtering operation on the AC signal Sac generated by the signal generator 22. For example, the filter 23 may include a low-pass filter and may convert the AC signal Sac in a sawtooth waveform into the AC signal Sac′ in a sine waveform. However, the type of the filter 23 is not limited thereto, and various types of filters may be employed depending on the design structure.

The calibrator 24 may generate the driving signal Vd by mixing the DC signal Sdc with the AC signal Sac′ supplied through the filter 23. For example, the calibrator 24 may be implemented as an operating amplifier capable of summing (or superimposing) the AC signal Sac′ and the DC signal Sdc, but is not limited thereto.

Consequently, an offset corresponding to the DC signal Sdc may be generated in the AC signal Sac′, and thus the driving signal Vd containing both the AC component and DC component may be generated.

Since the driving signal Vd includes all the characteristics of the AC signal Sac, the driving signal Vd may be set at a frequency in the range of 1 KHz to 1000 MHz and may also be set at a frequency in the range of 5 MHz to 15 MHz which is more suitable for the biofilm removal. In addition, the amplitude of the driving signal Vd may be set to in the range of 0.1 mV to 10V.

Referring to FIG. 6a, the calibrator 24 may receive the AC signal Sac′ having an amplitude of A volt (V) from the filter 23, and superimpose the DC signal Sdc of B volt (V) as shown in FIG. 6b on the AC signal Sac′, thereby generating a final driving signal Vd as shown in FIG. 6c.

In this case, the voltage value of the DC signal Sdc may be set equal to or greater than the amplitude of the AC signal Sac′. Accordingly, the voltage value of the driving signal Vd may be set to 0 or more.

Ultimately, the DC offset value of the driving signal Vd may be set to be equal to or greater than the amplitude of the driving signal Vd.

If the DC offset value of the driving signal Vd is less than a value of the amplitude of the driving signal Vd, it results in an interval where the voltage of the drive signal Vd has a negative value, and the voltage in this interval has the negative value, causing a loss of electrical energy to occur.

However, when the DC offset value of the driving signal Vd is set equal to or greater than the amplitude of the driving signal Vd as in the embodiment of the present invention, it is possible to minimize the loss of electrical energy because the voltage of the driving signal Vd is always zero or greater.

Meanwhile, the DC signal Sdc may be generated by the voltage divider 25. For example, the voltage divider 25 may receive the output voltage Vo from the DC-DC converter 21, and perform voltage division on the output voltage Vo to generate the DC signal Sdc.

The voltage divider 25 may comprise a resistor string for dividing the output voltage Vo, but is not limited thereto.

When the output voltage Vo from the DC-DC converter 21 is suitable to be used directly to generate the driving signal Vd, the corresponding output voltage Vo may be used as the DC signal Sdc. In this case, the voltage divider 25 may be omitted and the output voltage Vo of the DC-DC converter 21 may be inputted to the calibrator 24.

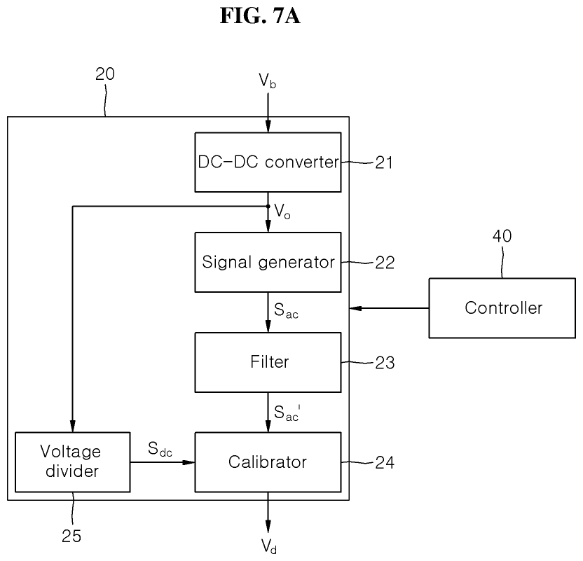

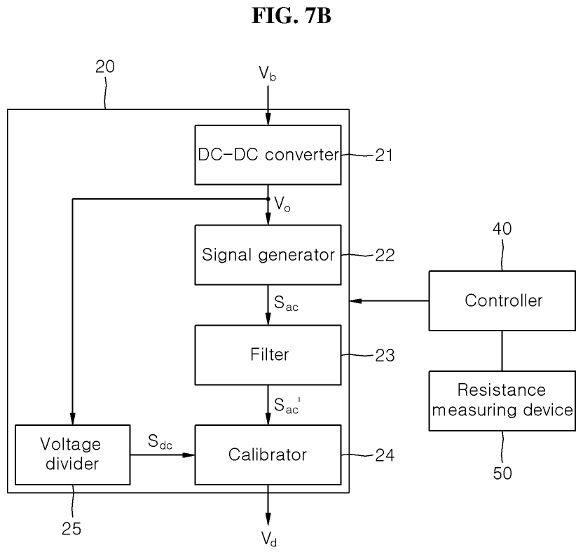

FIG. 7a is a diagram illustrating a controller and a signal supply module according to an embodiment of the present invention, and FIG. 7b is a diagram illustrating a resistance measuring device, a controller, and a signal supply module according to an embodiment of the present invention.

Referring to FIG. 7a, the signal supply module 20 according to an embodiment of the present invention may modify at least one of the characteristic of the driving signal Vd in response to an external input. For this purpose, a controller 40 may further be installed for controlling the signal supply module 20 in response to the external input (e.g., the user input).

For example, the characteristic of the driving signal Vd may include the amplitude and the DC offset of the driving signal Vd.

In other words, by adjusting at least one of the amplitude and DC offset of the driving signal Vd, the user may set an optimal driving signal Vd for biofouling prevention of the ship 1, and this enables the biofilm to be managed taking into account the characteristics for the area-specific of the ship.

In this case, the user's input method for controlling the characteristics of the driving signal Vd may be set in various ways. For example, the user may control the signal supply module 20 through a separate terminal (not shown) or a separate input device (not shown), which may be provided on the biofouling preventing device 10 for ships.

When setting information for the driving signal Vd is inputted by the user, the controller 40 may control the signal supply module 20 to provide the driving signal Vd having amplitude and DC offset value corresponding to the inputted setting information.

The controller 40 may modify the amplitude of the AC signal Sac by controlling the signal generator 22. In addition, the controller 40 may adjust the voltage value of the DC signal Sdc by controlling the DC-DC converter 21 and/or the voltage divider 25. Accordingly, the characteristic of the driving signal Vd may finally be modified.

In this case, the controller 40 may control the voltage divider 25 to set the voltage value of the DC signal Sdc to be equal to or greater than the amplitude of the AC signal Sac′, and thus the voltage value of the driving signal Vd may be set to be equal to or greater than zero.

Referring now to FIG. 7b, a resistance measuring device 50 may be further installed to measure the resistance of the electrode 11.

The resistance of the electrode 11 may be changed depending on the extent to which the biofouling occurs, and the controller 40 may control the characteristic of the driving signal Vd in response to the resistance value measured by the resistance measuring device 50. In other words, the extent to which biofouling occurs may be detected by changes in the resistance of the electrode 11, and at least one of the amplitude and the DC offset of the driving signal Vd may be adjusted to reflect the same, thereby increasing the removal effect of the biofouling.

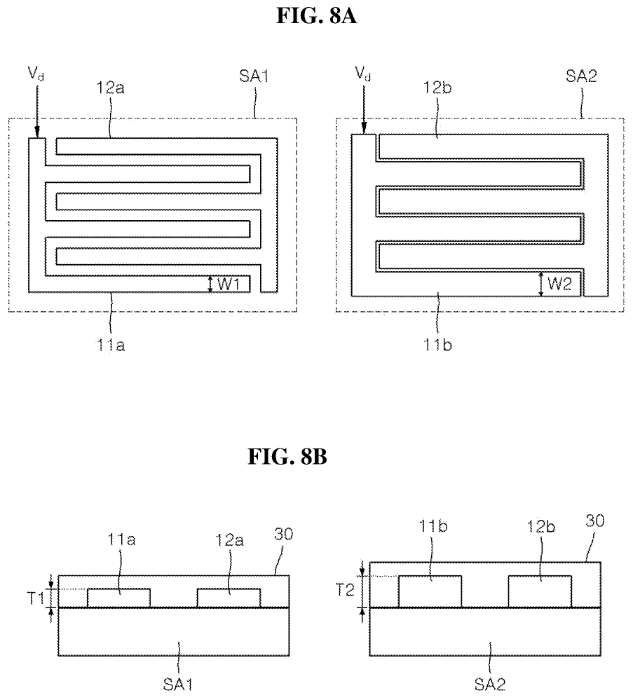

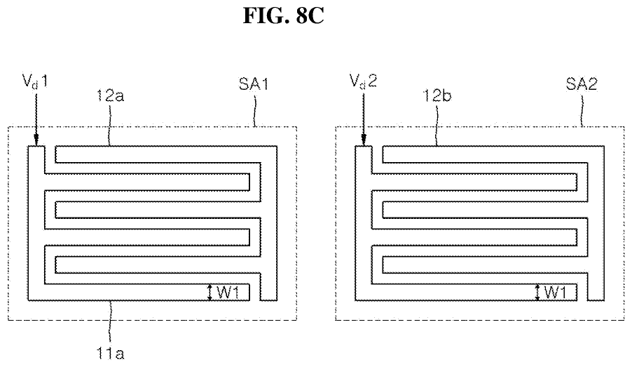

FIG. 8a is a diagram illustrating a first surface region and a second surface region of an embodiment of the present invention, FIG. 8b is a diagram illustrating a partial cross-section of the first surface region and the second surface region illustrated in FIG. 8a, and FIG. 8c is a diagram illustrating a first surface region and a second surface region of another embodiment of the present invention.

The surface of the ship 1 may be divided into a number of surface areas, and the extent to which bviceiofouling occurs may be different depending on the location, shape, characteristic, etc. of each surface area. In this case, an optimized biofouling management may be achieved on an area-specific basis by controlling the physical and/or electrical characteristic of the electrodes located on each surface area differently, rather than by applying the same approach to each surface area.

Referring to FIGS. 8a and 8b, the physical characteristics of a first electrode 11a disposed on a first surface area SA1 and a second electrode 11b disposed on a second surface area SA2 may be set differently.

For example, a physical characteristic of each electrode may include at least one of a width, a thickness, and a density of that electrode.

Specifically, the width W2 of the second electrode 11b may be set larger relative to the width W1 of the first electrode 11a, and the thickness T2 of the second electrode 11b may be set larger relative to the thickness T1 of the first electrode 11a.

Further, the density of the second electrode 11b may be set to be greater relative to than the density of the first electrode 11a. That is, when the areas of the first surface area SA1 and the second surface area SA2 are the same, the area ratio that the second electrode 11b occupies within the second surface area SA2 may be set to be larger than the area ratio that the first electrode 11a occupies within the first surface area SA1.

In the first surface area SA1, a first auxiliary electrode 12a serving as a negative electrode or ground electrode may be disposed together with the first electrode 11a supplied with the driving signal Vd, and in the second surface area SA2, a second auxiliary electrode (12b) serving as a negative electrode or ground electrode may be disposed together with the second electrode 11b supplied with the driving signal Vd.

When only the physical characteristics of the first electrode 11a and the second electrode 11b are to be set differently, the respective driving signals Vd supplied to the first electrode 11a and the second electrode 11b may be set the same.

In addition, the physical characteristics of the first auxiliary electrode 12a and the second auxiliary electrode 12b may also be set differently, and the physical characteristics of the first electrode 11a and the first auxiliary electrode 12a and/or the physical characteristics of the second electrode 11b and the second auxiliary electrode 12b may be set differently as needed.

Referring to FIG. 8c, the first electrode 11a disposed on the first surface area SA1 and the second electrode 11b disposed on the second surface area SA2 may be set to have different electrical characteristics.

When it is desired to set the electrical characteristics of the first electrode 11a and the second electrode 11b differently, the characteristics of the first driving signal Vd1 supplied to the first electrode 11a and the characteristic of the second driving signal Vd2 supplied to the second electrode 11b may be set differently.

For example, the electrical characteristic may include at least one of an amplitude and a DC offset of the driving signals Vd1 and Vd2 supplied to the respective electrodes.

Specifically, the amplitude of the second driving signal Vd2 may be set to be greater relative to the amplitude of the first driving signal Vd1. Similarly, the DC offset of the second driving signal Vd2 may be set to be greater relative to the DC offset of the first driving signal Vd1.

On the other hand, FIGS. 8a and 8b illustrate cases in which the physical characteristics of the respective electrodes 11A and 11B are set to be different, and FIG. 8c illustrates a case in which the electrical characteristics of the respective electrodes 11A and 11B are set to be different, but the physical characteristics and the electrical characteristics of the respective electrodes 11A and 11B may be set to be different at the same time.

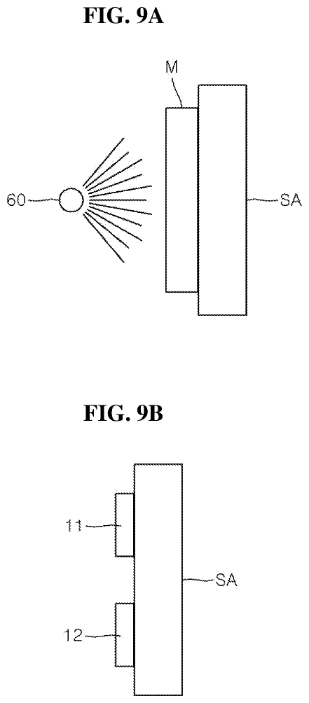

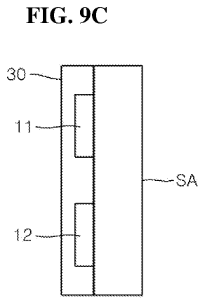

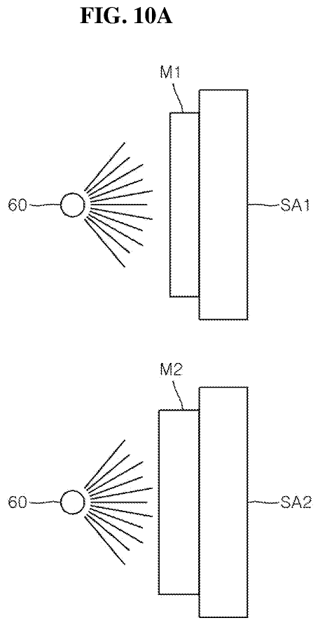

FIGS. 9a to 9c are diagrams illustrating a method of manufacturing a biofouling preventing device for ships according to an embodiment of the present invention.

Referring to FIG. 9a, a step of placing a mask M on the surface area SA on the hull 2 may first be performed.

Subsequently, a step of supplying conductive material to the side of the mask M by way of using a material supply device 60 may be performed to form the electrode 11 patterned in a predetermined shape on the surface area SA (see FIG. 9b).

For example, the material supply device 60 may be implemented as a device for spraying conductive material onto the mask M, or as a device for supplying conductive material in a printing manner. Additionally, a curing process may further be performed on the conductive material attached to the surface area SA, and the auxiliary electrode 12 may also be formed by the same shadow mask method as the electrode 11.

Referring to FIG. 9c, a step of forming a protective layer 30 on top of the electrode 11 and the auxiliary electrode 12 may be performed. The protective layer 30 may be non-conductive and may serve to protect the electrode 11 and the auxiliary electrode 12 covered with the protective layer.

Further, a step of installing a signal supply module 20 for supplying the driving signal Vd to the electrode 11 may be performed.

The signal supply module 20 may be installed at a predetermined distance from the surface area SA, and may be electrically connected to the electrode 11 and the auxiliary electrode 12 via separate wirings 15 and 16 (see FIG. 2).

These wirings 15 and 16 may also be formed through a patterning process using shadow masks. For this purpose, a step of placing wiring masks on wiring areas and supplying conductive material to the sides of the wiring masks using the material supply device 60 may be further performed, thereby forming the wirings 15 and 16 patterned in a predetermined shape on the respective wiring areas.

The use of the shadow mask method described above may reduce manufacturing time and cost by facilitating the formation of large-area electrodes, and may improve a stability of ships by eliminating the need for separate holes during manufacturing.

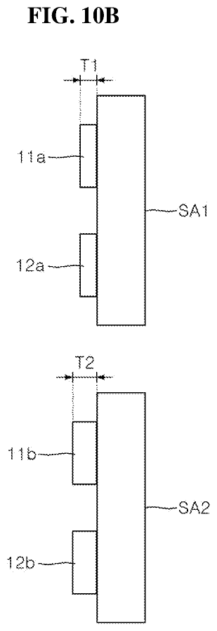

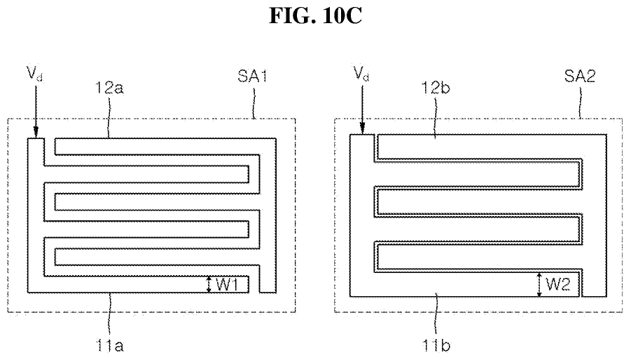

FIGS. 10a to 10c are diagrams illustrating a method of manufacturing a biofouling preventing device according to another embodiment of the present invention.

Referring to FIG. 10a, a step of placing a first mask M1 on a first surface area SA1 of the hull 2 may be performed. Subsequently, a step of supplying conductive material to the side of the first mask M1 using the material supply device 60 may be performed to form a first electrode 11a patterned in a predetermined shape on the first surface area SA1 (see FIG. 10b). Further, in this step, a first auxiliary electrode 12a may be formed together with the first electrode 11a.

A step of placing a second mask M2 on a second surface area SA2 of the hull 2 may be performed. Thereafter, a step of providing conductive material to the side of the second mask M2 using the material supply device 60 may be performed to form a second electrode 12a patterned in a predetermined shape on the second surface area SA2 (see FIG. 10b). Further, in this step, a second auxiliary electrode 12b may be formed together with the second electrode 11b.

Further, a step of installing the signal supply module 20 for supplying a driving signal Vd to each of the electrodes 11a, 11b may be performed.

The first electrode 11a and the second electrode 11b formed through the above-described processes may have at least one of physical characteristics and electrical characteristics that are set to be different.

For example, a physical characteristic of each electrode may include at least one of a width, a thickness, and a density of that electrode.

Specifically, the width W2 of the second electrode 11b may be set to be large relative to the width W1 of the first electrode 11a. Further, the thickness T2 of the second electrode 11b may be set larger relative to the thickness T1 of the first electrode 11a, in which case the thickness of the second mask M2 may be set larger than the first mask M1.

Further, the density of the second electrode 11b may be set to be greater relative to than the density of the first electrode 11a. That is, when the areas of the first surface area SA1 and the second surface area SA2 are the same, the area ratio that the second electrode 11b occupies within the second surface area SA2 may be set to be larger than the area ratio that the first electrode 11a occupies within the first surface area SA1.

When only the physical characteristics of the first electrode 11a and the second electrode 11b are to be set differently, the respective driving signals Vd supplied to the first electrode 11a and the second electrode 11b may be set the same.

In addition, the physical characteristics of the first auxiliary electrode 12a and the second auxiliary electrode 12b may also be set differently, and the physical characteristics of the first electrode 11a and the first auxiliary electrode 12a and/or the physical characteristics of the second electrode 11b and the second auxiliary electrode 12b may be set differently as needed.

Although not shown separately, the electrical characteristics of the first electrode 11a disposed on the first surface area SA1 and the second electrode 11b disposed on the second surface area SA2 may be set to be different.

When it is desired to set the electrical characteristics of the first electrode 11a and the second electrode 11b differently, the characteristics of the first driving signal Vd1 supplied to the first electrode 11a and the characteristic of the second driving signal Vd2 supplied to the second electrode 11b may be set differently.

For example, the electrical characteristic may include at least one of an amplitude and a DC offset of the driving signals Vd1 and Vd2 supplied to the respective electrodes.

Specifically, the amplitude of the second driving signal Vd2 may be set to be greater relative to the amplitude of the first driving signal Vd1. Similarly, the DC offset of the second driving signal Vd2 may be set to be greater relative to the DC offset of the first driving signal Vd1.

Those skilled in the art to which the present invention pertains will understand that the present invention may be embodied in other specific forms without changing its technical idea or essential features. Therefore, the embodiments described above should be understood in all respects as illustrative and not restrictive. The scope of the present invention is defined by the following claims rather than by the above detailed description, and all changes or modifications derived from the claims and their equivalents should be construed as being within in the scope of the present invention.

1. A biofauling preventing device for ships comprising:
an electrode disposed on a surface area of a hull and configured to supply with a driving signal to provide electromagnetic waves corresponding to the driving signal; and
a signal supply configured to supply the driving signal to the electrode.

2. The biofauling preventing device for ships of claim 1, wherein the driving signal is generated by mixing an AC signal and a DC.

3. The biofauling preventing device for ships of claim 2, wherein the signal supply is configured to modify at least one of the characteristics of the driving signal in response to an external input.

4. The biofauling preventing device for ships of claim 3, wherein the characteristics of the driving signal include an amplitude and a DC offset.

5. The biofauling preventing device for ships of claim 1, further comprising:
a protective layer disposed on the electrode.

6. The biofauling preventing device for ships of claim 1, wherein the surface area includes a first surface area and a second surface area, and
the electrode includes a first electrode disposed on the first surface region and a second electrode disposed on the second surface region.

7. The biofauling preventing device for ships of claim 6, wherein the first electrode has a width different from the second electrode.

8. The biofauling preventing device for ships of claim 6, wherein the signal supply supplies a first driving signal and a second driving signal, which are a mixture of the AC signal and the DC signal, to the first electrode and the second electrode, respectively, and
at least one of an amplitude and a DC offset of the first driving signal is set to be different from at least one of a corresponding amplitude and a DC offset of the second driving signal.

9. The biofauling preventing device for ships of claim 2, further comprising:
a resistance measuring device configured to measure the resistance of the electrode,
the signal supply modify at least one of an amplitude and a DC offset of the drive signal in response to a resistance value measured by the resistance measuring device.

10. A method of manufacturing a biofouling preventing device for a ship, the method comprising:
placing a mask on a surface area of a hull;
supplying a conductive material to the side of the mask to form an electrode patterned in a predetermined shape on the surface area; and
forming a protective layer on the electrode.

11. The method of manufacturing a biofouling preventing device for a ship of claim 10, further comprising:
installing a signal supply configured to supply a driving signal to the electrode,
wherein the driving signal is generated by mixing an AC signal and a DC signal.

12. A method of manufacturing a biofouling preventing device for a ship, the method comprising:
placing a first mask on a first surface area of a hull;
supplying a conductive material to the side of the first mask to form a first electrode patterned in a predetermined shape on the first surface area;
placing a second mask on a second surface area of the hull; and
supplying a conductive material to the side of the second mask to form a second electrode patterned in a predetermined shape on the second surface area,
wherein the first electrode and the second electrode have at least one of a physical characteristic and an electrical characteristic that are set to be different.

13. The method of manufacturing a biofouling preventing device for a ship of claim 12, wherein the physical characteristic includes at least one of a width, a thickness, and a density of the first electrode and the second electrode, and
the electrical characteristic includes at least one of an amplitude and a DC offset of the driving signal supplied to the first electrode and the second electrode.
